(19) United States
(12) Patent Application Publication
(10) Pub. No.: US 2008/0153786 A1
(43) Pub. Date: Jun. 26, 2008

(54) Controlled release of drugs into/through the skin

(75) Inventors: Philippe Andres, Peymeinade (FR); Claire Mallard, Mougins (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 12/000,187

(22) Filed: Dec. 10, 2007

Related U.S. Application Data
(63) Continuation of application No. PCT/EP2006/005831, filed on May 22, 2006.

(51) Int. Cl. A61K 31/59; A61P 17/02; A61P 17/06

(52) U.S. Cl. 514/167

(57) ABSTRACT

The slow and sustained, controlled release of a drug into and through the skin includes topically applying onto the skin of an individual in need of such treatment, a composition containing at least one solubilized drug, at least one film-forming silicone, and at least one volatile solvent.

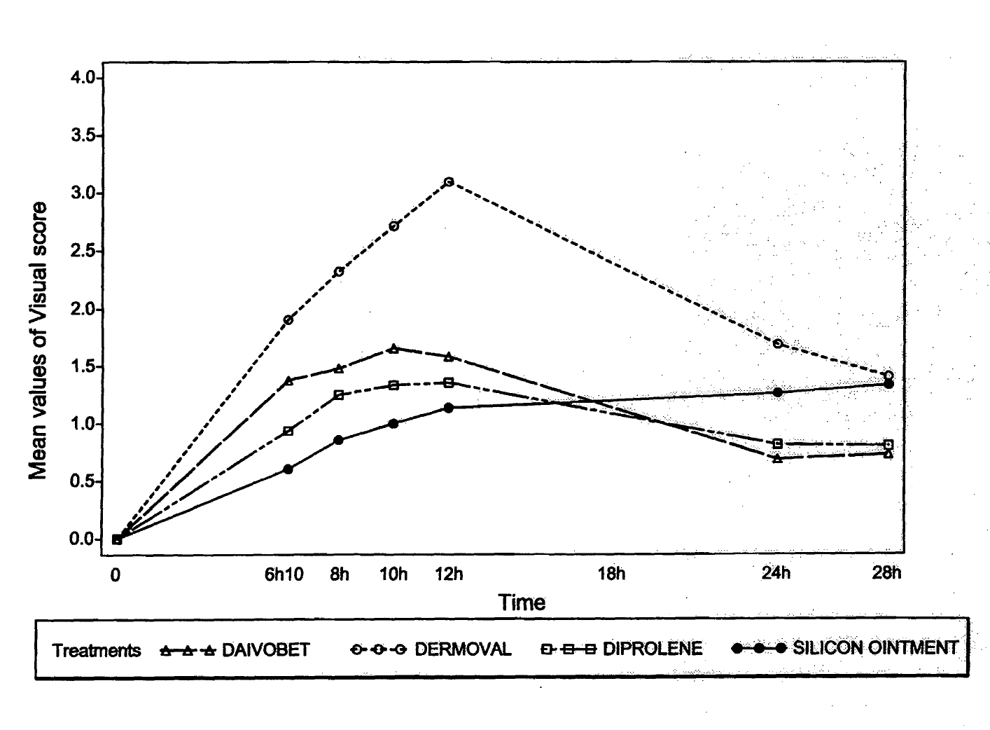

### CROSS-REFERENCE TO PRIORITY/PCT APPLICATIONS

This application claims priority of Provisional Application No. 60/689,282, filed Jun. 10, 2005, and is a continuation of PCT/EP 2006/005831, filed May 22, 2006 and designating the United States, published in the English language as WO 2006/131401 A2 on Dec. 14, 2006, each hereby expressly incorporated by reference in its entirety and each assigned to the assignee hereof.

### BACKGROUND OF THE INVENTION

1. Technical Field of the Invention

The present invention relates to the field of drug formulation for topical administration.

2. Description of Background and/or Related and/or Prior Art

The poor penetration of drugs into the skin (and, partially, the permeation across the Stratum corneum) often limits the efficacy of topical formulations. Basically, skin penetration can be enhanced by the following strategies: (i) increasing drug diffusivity in the skin; (ii) increasing drug solubility in the skin, and/or (iii) increasing the degree of saturation of the drug in the formulation. However supersaturated formulations, in which the degree of saturation of the drug is increased compared to conventional formulations, are often unstable, mainly because of crystallization of the drug.

### SUMMARY OF THE INVENTION

The present invention features the controlled release of a drug into and through the skin, which comprises topically administering a composition that contains at least one solubilized drug, a film-forming silicone, and at least one volatile solvent.

More particularly, this invention features a method wherein the drug penetrates the upper layers of the skin that serve as a reservoir wherein the drug concentrates before being released to the dermis.

For example, the drug may be vitamin D or a vitamin D analogue, such as calcitriol, or a corticosteroid, such as clobetasol or clobetasol-17-propionate, whether alone or in combination.

It has now been found that topical compositions that comprise at least one solubilized drug, a film-forming silicone, and at least one volatile solvent permit the controlled release of the drug through skin, while showing a good stability. The release of the drug is slow and sustained, which makes it possible to lower the dosage. The drug can thus be administered at a dosage that is lower than the dosage used for compositions comprising the same drug, but devoid of the film-forming silicone and the volatile solvent.

The drug penetrates into the skin according to a specific zero-order kinetics, which means that the drug concentration exhibits a linear variation vs. time, and that the penetration is constant and sustained. The drug is first maintained within the upper layers of the skin, that is to say, the layers of:

Stratum corneum,

Stratum lucidum,

Stratum granulosum, and

Stratum germinativum (including Stratum spinosum and Stratum basale).

### BRIEF DESCRIPTION OF THE DRAWINGS

The FIGURE of Drawing shows the results of a blanching test after topical application of various compositions as described in Example 2.

### DETAILED DESCRIPTION OF BEST MODE AND SPECIFIC/PREFERRED EMBODIMENTS OF THE INVENTION

The release of the drug into the lower layers (i.e., dermis and hypodermis) is controlled by the in situ supersaturation of the drug. Supersaturation is achieved when the solvent evaporates after the composition is applied onto skin. This evaporation concentrates the drug in solution, which favors its penetration in the upper layers of the skin and creates a reservoir effect. In parallel, the silicone allows the control of the evaporation kinetics of the solvent and control of the crystallization of the drug, which also favors its penetration.

The compositions described herein comprise at least one drug, i.e., a pharmaceutically active or bioactive ingredient. In particular, same may comprise two drugs.

Examples of drugs of interest are vitamin D or a vitamin D analogue.

The term “vitamin D” means the various forms of vitamin D such as vitamin D₁, D₂, D₃ or vitamin D₄.

The term “vitamin D analogue” means the compounds that exhibit analogous biological properties compared to vitamin D, in particular with regard to the transactivation of the response elements of vitamin D (VDRE), such as an agonist or antagonist activity towards the vitamin D receptors. These compounds preferably are synthetic compounds that comprise the skeleton of vitamin D with modifications of lateral chains and/or that also comprise modifications within this skeleton. The analogues may comprise structural analogues, in particular biaromatic compounds.

Preferably, the vitamin D analogue is selected from the group consisting of calcitriol, calcipotriol, doxercalciferol, secalcitol, maxacalcitol, seocalcitol, tacalcitol, paricalcitol, falecalcitriole, 1α,24S-dihydroxy-vitamine D2, 1(S),3(R)-dihydroxy-20(R)-[((3-(2-hydroxy-2-propyl)-phenyl)-methoxy)-methyl]-9,10-seco-pregna-(Z),7(E),10(19)-triene and mixtures thereof. Most preferably, it is calcitriol.

Further examples of vitamin D analogues include those described in WO 02/34235, WO 00/64450, EP-1,124,779, EP-1,235,824, EP-1,235,777, WO 02/94754, WO 03/050067 and WO 00/26167. The compounds described in WO 00/26167 relate to structural analogues of vitamin D that show a selective activity on cell proliferation and differentiation without showing hypercalcemic activity.

Advantageously, the quantity of vitamin D or vitamin D analogue solubilized in the composition is from 0.00001 to 5% w/w, preferably from 0.0001 to 3% w/w and more preferably from 0.0003 to 1% w/w.

Another drug of interest, alone or in combination with vitamin D or the vitamin D analogue, is a corticosteroid.

The term “corticosteroid” means a topical steroid of group I, II, III or IV (strong or weak).

More particularly, it may be selected from the group consisting of betamethasone, clobetasol, clobetasone, desoximethasone, diflucortolon, diflorasone, fluocinonide, flumethasone, fluocinolon, fluticasone, fluprednidene, halcinonide, hydrocortisone, momethasone, triamcinolon, pharmaceutically acceptable esters or acetonides thereof, and mixtures thereof.

Examples of esters or acetonides include 17-valerate, 17-propionate, 17,21-dipropionate, acetonide, acetonide-21-N-benzoyl-2-methyl-β-alaninate, acetonide-21-(3,3-dimethylbutyrate) and 17-butyrate.

Most preferably, the corticosteroid is clobetasol or clobetasol-17-propionate.

Advantageously, the quantity of corticosteroid in a solubilized form in the composition is from 0.0001 to 1% w/w, more preferably from 0.0005 to 3% w/w, and more preferably from 0.001 to 0.1% w/w.

In a preferred embodiment, the active ingredients are solubilized in the same solvent or in several solvents.

The solvent is selected from among pharmaceutically acceptable compounds, i.e., compounds that are suitable for a topical application.

Preferred volatile solvents include alkanols, alkylglycols, alkylketones and/or alkyl esters wherein the alkyl moieties contain from 1 to 6 carbon atoms, preferably from 1 to 4 carbon atoms, such as ethanol, isopropanol, n-butanol, ethyl acetate, acetone, and mixtures thereof.

Preferably the volatile solvent is ethanol, especially when the drugs are calcitriol and clobetasol-17-propionate.

Advantageously, the quantity of solvent within a composition is from 1 to 50% w/w (based on the total weight of the composition), preferably from 2 to 40% w/w and more preferably from 5 to 20% w/w.

The film-forming silicone according to the invention preferably comprises at least one polyorganosiloxane elastomer.

The term “polyorganosiloxane elastomer” refers to any siloxane polymer, which is chemically crosslinked and which exhibits viscoelastic properties.

Examples of suitable polyorganosiloxane elastomers according to the invention are those described in U.S. Pat. Nos. 4,980,167 and 4,742,142. Such polyorganosiloxanes may especially be addition products (adducts) resulting from hydrosylation, and/or polymeric products deriving from the addition of (i) a polyorganosiloxane having unsaturated groups, such as vinyl or allyl groups, for example linked to at least one atom, and (ii) another silicone product able to be involved in the addition reaction, such as an organohydrogenopolysiloxane.

According to a specific embodiment, the polyorganosiloxane elastomer is present in at least one volatile silicone oil that is a linear or cyclic polyorganosiloxane oil having 2 to 10 silicon atoms.

The terms “polyorganosiloxane oils” refers to any silicone oil able to evaporate in contact of skin, mucosa or keratinic fibers, preferably with an evaporation duration of less than 1 hour, at ambient temperature and water atmospheric pressure.

Polyorganosiloxane oils according to the invention are, for example, linear or cyclic polyorganosiloxanes having 2 to 10 silicon atoms, optionally comprising alkyl or alkoxy groups having 1 to 22 carbon atoms. Silicone oils according to the invention advantageously exhibit a viscosity of at most 6.10⁻⁶ m²/s (6 centistokes).

Suitable volatile silicone oils especially include cyclomethicones and/or dimethicones of low molecular weight. In this event, cyclic polyorganosiloxanes, especially cyclic methoxylated organospolysiloxane, having a 4-membered to 12-membered siloxane ring such as octamethylcyclotetrasiloxane and decamethylcyclopentasiloxane, may be used. Other suitable volatile silicone oils are dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, and mixtures thereof.

A particularly suitable film-forming silicone according to the invention comprises a polyorganosiloxane elastomer in decamethyltetrasiloxane. In this embodiment, a preferred silicone product is the so-called “ST Elastomer 10®” of DOW CORNING, which is formulated in the form of a viscous and translucid gel.

According to another specific embodiment, the film-forming silicone employed in the method of the invention is a silicone product obtained by a crosslinking of:

(A) a polysiloxane having ≡SiH groups;

(B) an alpha, omega-diene;

(C) a polysiloxane having a low molecular weight,

in the presence of a catalyst.

In this embodiment, polysiloxane (A) advantageously comprises one or more compounds having one of the following formulae (A¹), (A²⁻¹) and (A²⁻²):

R₃¹⁴SiO(R¹⁵₂SiO)ₐ(R¹⁶HSiO)_{b}SiR₃¹⁴ (A¹)

HR₂¹⁴SiO(R¹⁵₂SiO)ₐ(R¹⁶HSiO)_{b}SiR₂¹⁴H (A²⁻¹),

HR₂¹⁴SiO(R¹⁵₂SiO)_{c}SiR₂¹⁴H (A²⁻²)

wherein:

R¹⁴, R¹⁵ and R¹⁶, which may be identical or different, are each an alkyl radical having 1 to 6 carbon atoms;

a is an integer having a value of 0 to 250,

b is an integer having a value of 1 to 250; and

c is an integer having a value of 0 to 250.

Preferably, polysiloxane (A) contains compounds of above formulae (A²⁻¹) and/or (A²⁻²), preferably together with compounds of formula (A¹), with a molar ratio (A²⁻¹+A²⁻²):(A¹) preferably between ranging from 0 to 20, especially from 0 to 5.

Alpha, omega-diene (B) is a compound of formula CH₂═CH(CH₂)_{d}CH═CH₂, wherein d is an integer having a value of 1 to 20.

Representative examples of suitable alpha, omega-dienes are especially 1,4-pentadiene, 1,5-hexadiene, 1,6-heptadiene, 1,7-octadiene, 1,8-nonadiene, 1,9-decadiene, 1,11-dodecadiene, 1,13-tetradecadiene, et 1,19-eicosadiene.

Polysiloxane (C) may especially include, whether alone or in combination:

(C1) linear, branched, or cyclic volatile methylsiloxanes, for example:

volatile methoxysiloxanes selected from among hexamethyldisiloxane, octamethyltrisiloxane, decamethyltetrasiloxane, dodecamethylpentasiloxane, tetradecamethylhexasiloxane, and/or hexadecamethylheptasiloxane;

cyclic volatiles methylsiloxanes such as hexamethylcyclotrisiloxane, octamethylcyclotetrasiloxane, decamethylcyclopentasiloxane, and/or dodecamethylcyclohexasiloxane.

branched volatile methylsiloxanes, such as heptamethyl-3-[(trimethylsilyl)oxy]-trisiloxane, hexamethyl-3,3,bis[(trimethylsilyl)oxy]-trisiloxane, and/or pentamethyl[(trimethylsilyl)oxy]-cyclotrisiloxane;

(C2) alkyl- and/or aryl-siloxanes, which are linear, or cyclic, and which are volatile or non-volatile,
especially low molecular weight, non-volatile, compounds having a viscosity of about 100 to 1000 mm²/s (centistokes), especially those having the following formula:

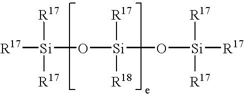

wherein:

e has a value preferably of 80 to 375,

R¹⁷ et R¹⁸ are alkyl radicals having 1 to 20 carbon atoms, or an aryl group such as a phenyl,

for example polydimethylsiloxanes, polydiethylsiloxanes, polymethylethylsiloxanes, polymethylphenylsiloxanes and/or polydiphenylsiloxanes;
(C3) functionalized siloxanes, which are linear, or cyclic, especially fluid siloxanes, for example functionalized with groups selected from among acrylamides, acrylates, amides, amino, carbinol, carboxy, chloroalkyles, epoxy, glycol, cetal, mercapto, methylester, perfluoro and silanol.

Preferably, Polysiloxane (C) is a low molecular weight silicone oil selected from volatile methylsiloxanes, of low molecular weight, which are linear or cyclic.

Other polysiloxanes suitable for use as film-forming silicones according to the invention are silicone polymers having an average molecular weight of at least 10,000 (e.g. from 10,000 to 10,000,000). Examples of such polysiloxanes include copolymers of crosslinked siloxanes, especially copolymers of dimethicone or dimethicone derivatives, for example siloxane stearyl methyl-dimethyl copolymers (such as <<Gransil SR-CYC®>> of Grant Industries), copolymers of the type of the <<Polysilicone-11®>> (crosslinked elastomer silicone formed by reaction of a vinyl-terminated silicone with methylhydrodimethylsiloxane in the presence of cyclomethicone), crosslinked cetearyl dimethicone/vinyl dimethicone copolymers (namely copolymers of cetearyl dimethicone crosslinked with a vinyl dimethyl polysiloxane), crosslinked dimethicone/phenyl vinyl dimethicone copolymers (namely dimethylpolysiloxane copolymers crosslinked with phenyl vinyl dimethylsiloxane), and crosslinked dimethicone/vinyl dimethicone copolymers (namely dimethylpolysiloxane copolymers crosslinked with vinyl dimethylsiloxane).

Silicones formulated as a gel may be obtained especially from Grant Industries. Examples of such gels especially include:

mixtures of cyclomethicone and polysilicone-11, such as commercial product <<Gransil GCM5®>>,

mixtures of cyclotetrasiloxane and polysilicone-11, such as commercial product <<Gransil PS-4®>>,

mixtures of cyclopentasiloxane and polysilicone-11 such as commercial product <<Gransil PS-5®>>,

mixtures of cyclopentasiloxane, dimethicone and polysilicone-11, such as commercial product <<Gransil DMCM-5®>>,

mixtures of cyclotetrasiloxane, dimethicone and polysilicone-11, such as commercial product <<Gransil DMCM-4®>>,

mixtures of polysilicone-11 and isododecane such as commercial product <<Gransil IDS®>>, and

mixtures of cyclomethicone, polysilicone-11 and phytosphingosine, such as commercial product <<Gransil SPH®>>.

Other examples are gels of crosslinked polymers of cyclopentasiloxane and dimethicone/vinyl dimethicone, such as <<SFE839®>> of the General Electric Company. Yet other silicone gels are those commercialized by the Shin-Etsu Company under references KSG-15, KSG-16 and KSG-17.

According to another specific embodiment, the composition employed in the method of the invention is advantageously free from polyorganosiloxane having alkyl groups.

Whatever its exact nature, the film-forming silicone of the method of the invention is advantageously present in the composition at a concentration of 20 to 90% weight based on the total weight of the composition, preferably of from 30 to 80%.

The compositions described herein may further contain an oily additive, such as isopropyl palmitate, dicaprilic ether, dimethicone, or mixtures thereof.

The compositions described herein may also contain flavor-enhancing agents, preservatives such as para-hydroxybenzoic acid esters, stabilizing agents, moisture regulators, pH regulators, osmotic pressure modifiers, emulsifying agents, UV-A and UV-B screening agents, and antioxidants such as α-tocopherol, butylhydroxyanisole or butylhydroxytoluene, superoxide dismutase, ubiquinol, or certain chelating agents.

Preferably, the composition is in form of a cream, a gel, an ointment or a pomade.

Preferably, the composition is substantially free of water, i.e., it contains less than 5% w/w of water, preferably less than 3%, most preferably 0% of water.

Preferred compositions comprise:

isopropyl palmitate

cyclopentasiloxane

cyclomethicone 5/dimethicone crosspolymer

calcitriol

clobetasol-17-proprionate

ethanol.

In a preferred embodiment, the composition comprises:

isopropyl palmitate 0.5-2%

cyclopentasiloxane 10-20%

cyclomethicone 5/dimethicone crosspolymer 70-80%

calcitriol 0.0001-0.0005%

clobetasol-17-proprionate 0.01-0.05%

ethanol 5-10%.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

### Example 1

### Preparation of a Controlled-Release Formulation

The process described below is a general manufacture process of a silicone ointment that comprises a vitamin D analogue and a corticosteroid. The process is carried out at room temperature, from 20° C. to 25° C.

First Step: Preparation of the Phase that Comprises the Silicone (Phase I):

The ingredients of phase I (“Elastomer ST 10®”, silicone oil and oily additive) are weighed in a vessel. The mixture is homogenized until obtention of a homogenous gel.

Second Step: Preparation of the Phase that Comprises the Active Ingredients (Phase II):

A parent solution is prepared, that comprises a vitamin D analogue in an appropriate solvent, and an anti-oxidant. The solution is stirred until solubilization of the active ingredient.

The corticosteroid is weighed and introduced into the solvent. The solution is stirred until solubilization of the active ingredient.

The two active phases are incorporated in phase I under stirring. The mixture is homogenized.

When the solvent is the same for the two active ingredients, the corticosteroid is added to the parent solution of vitamin D analogue.

TABLE 1
Composition

| Ingredients | Quantities in % (w/w) |
|---|---|
| PHASE I: | 1 |
| ISOPROPYL PALMITATE¹ | |
| (oily additive) | |
| CYCLOPENTASILOXANE² | 16 |
| (solvent) | |
| CYCLOMETHICONE 5/ | 74.95 |
| DIMETHICONE CROSSPOLYMER | |
| (silicone agent³) | |
| PHASE II: | 0.04 |
| BUTYLHYDROXYTOLUENE | |
| (anti-oxidant) | |
| CALCITRIOL | 0.0003 |
| (active ingredient) | |
| CLOBETASOL PROPIONATE | 0.025 |
| (active ingredient) | |
| ABSOLUTE ETHANOL | 8 |
| (solvent) | |

¹Crodamol IPP ®
²Mirasil CM5 ®
³Elastomer ST 10 ®

### Example 2

### Sustained-Release of the Drug

The objective of this study was to compare a fixed-combination of calcitriol 3 μg/g and clobetasol propionate 250 μg/g (composition of example 1) by evaluation of its blanching capacity to three marketed corticosteroids formulations:

Dermoval® (Temovate®) cream (clobetasol propionate 500 μg/g)

Diprolene® cream (betamethasone dipropionate 500 μg/g)

Daivobet® ointment (fixed-combination containing calcipotriol 50 μg/g and betamethasone dipropionate 500 μg/g).

The creams of reference (Dermoval®, Diprolene®, Daivobet®) above do not contain a combination of silicone and volatile solvent.

Methodology:

This study was conducted as a single center, investigator masked, active controlled, intra-individual comparison.

The tested products were randomly allocated to pre-marked 2.2 cm diameter sites on forearms. Applications were performed by a trained research assistant out of the sight of the blanching evaluators. The study products were administrated as six hours non occlusive application.

Visual and chromametric evaluations of vasoconstriction were made within 30 minutes before product application, and 10 minutes, 2 hours, 4 hours, 6 hours, 18 hours and 22 hours after removal of the excess (removal took place 6 hours after study products application). Assessment of blanching visual scores (primary pharmacodynamics variable) was performed by two independent trained evaluators, using a 5-point scale (0: no blanching to 4: maximal blanching). Chromametric evaluation (secondary pharmacodynamics variable) was based on chromametric parameters (a* and L* value), using a ChromaMeter Minolta CR 300.

Safety assessment was conducted for all subjects at every visit after enrolment in the study. The primary parameter for the safety measurement was the record of adverse events.

Visual scoring was to be made independently by two experienced evaluators using the following 5 point-scale:

0 No change in skin color

1 Slight (barely visible) blanching

2 Obvious blanching

3 Intense blanching

4 Maximal blanching considered being

For visual scores, the analyzed variable was the mean of the two evaluators' scores on each site. The area under the curve was calculated by subject and by treatment from T0 (before application) up to T28h (22 hours after product removal). The chromametric variables a* and L* were adjusted according to their baseline value before application: —Δa* and ΔL*. The area under the curve was calculated by subject and by treatment from T0 (before application up to T28h (22 hours after product removal). The areas under the curve were submitted, by parameter, to analyses of variance including subject, zone and treatment as factors in the model.

The treatments were compared and classified using a Tukey multiple comparison test, which was performed at the 5% two sided significance level.

Results:

Twenty-four (2 male and 22 female healthy subjects aged 20.4 to 42.3 years) were enrolled in the study. Twenty-four subjects completed the study according to the protocol. None of them was excluded from the analysis.

Regarding the evaluation of the blanching visual scores (based on a 5-point scale), the analyzed variable was the area under the curve (AUC) of mean of the two evaluators' scores on each site. This AUC was calculated by subject and by treatment from T0 (before application) up to T28h (22 hours after product removal). These data are summarized in Table 2 below:

| | | | | | silicone |
|---|---|---|---|---|---|
| | | Daivobet ® | Dermoval ® | Diprolene ® | ointment |
| AUC | n | 24 | 24 | 24 | 24 |
| | Mean | 29.69 | 55.46 | 26.40 | 26.75 |
| | ±SEM | ±2.76 | ±2.66 | ±2.73 | ±2.47 |
| | Median | 30.84 | 58.51 | 25.54 | 27.93 |
| | (Min, | (0.48, | (32.25, | (6.23, | (6.00, |
| | Max) | 53.06) | 76.25) | 48.79) | 56.88) |

Based on the visual scores of blanching (primary pharmacodynamics variable), investigational products were classified in two separate groups with a significantly different vasoconstriction activity, as follows:

Dermoval® cream>

silicone ointment, Daivobet® ointment, Diprolene® cream.

However a very specific vasoconstriction profile was observed with the silicone ointment demonstrating a very slow release. The maximal effect was not reached after T0+22 hours, that is 28 hours after product application. The AUC of this product is therefore not complete and cannot be appropriately compared to the other products for which entire AUC could be calculated.

The chromametric parameters L* and a* supported the results obtained from visual scores.

In terms of safety analysis, neither treatment-related adverse events nor serious adverse events were reported. Only one unrelated adverse event (common cold) was reported during the study. All tested products were therefore considered well-tolerated.

The FIGURE of Drawing shows the results of the blanching test, presented as mean values of visual core across time after topical application of Dermoval®, Diavobet® creams or a silicone ointment, as described herein.

Conclusion:

The release of clobetasol from the silicone ointment is continuously increasing with the maximal effect of vasoconstriction not reached after 28 hours.

### Example 3

### Distribution of the Drug

Clobetasol-17-propionate was shown to accumulate in the Stratum corneum 16 hours after application on a human skin (Franz' cells).

TABLE 3

| | % Applied Dose | | | | |
|---|---|---|---|---|---|
| | Stratum | | | | |
| | corneum/ | | Absorbed | Dermal | Mass |
| Formulations | Epidermis | Dermis | dose | delivery | balance |
| Temovate ® | 5.33 ± 0.54 | 2.62 ± 0.38 | 0.48 ± 0.01 | 8.43 ± 0.79 | 98.76 ± 2.33 |
| Cream | | | | | |
| Silicone | 8.24 ± 1.28 | 1.12 ± 0.18 | 0.59 ± 0.01 | 9.96 ± 1.36 | 97.82 ± 3.66 |
| Ointment | | | | | |

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A method for the slow and sustained, controlled release of a drug into and through the skin, which comprises topically applying onto the skin of an individual in need of such treatment, a pharmaceutically active composition comprising at least one solubilized drug, at least one film-forming silicone, and at least one volatile solvent.

2. The method as defined by claim 1, wherein the drug is administered at a dosage that is lower than the dosage in compositions comprising the same drug, but devoid of the film-forming silicone and the volatile solvent.

3. The method as defined by claim 1, wherein the composition comprises at least two drugs.

4. The method as defined by claim 1, wherein the composition comprises solubilized vitamin D or a vitamin D analogue.

5. The method as defined by claim 4, wherein the composition comprises a vitamin D analogue selected from the group consisting of calcitriol, calcipotriol, doxercalciferol, secalcitol, maxacalcitol, seocalcitol, tacalcitol, paricalcitol, falecalcitriole, 1α,24S-dihydroxy-vitamine D2, 1(S),3(R)-dihydroxy-20(R)-[((3-(2-hydroxy-2-propyl)-phenyl)-methoxy)-methyl]-9,10-séco-pregna-(Z),7(E),10(19)-triene and mixture thereof.

6. The method as defined by claim 5, wherein the vitamin D analogue is calcitriol.

7. The method as defined by claim 1, wherein the composition comprises a solubilized corticosteroid.

8. The method as defined by claim 7, wherein the corticosteroid is selected from the group consisting of betamethasone, clobetasol, clobetasone, desoximethasone, diflucortolon, diflorasone, fluocinonide, flumethasone, fluocinolon, fluticasone, fluprednidene, halcinonide, hydrocortisone, momethasone, triamcinolon, pharmaceutically acceptable esters or acetonides thereof, and mixtures thereof.

9. The method as defined by claim 7, wherein the composition comprises the esters or acetonides selected from the group consisting of 17-valerate, 17-propionate, 17,21-dipropionate, acetonide, acetonide-21-N-benzoyl-2-methyl-β-alaninate, acetonide-21-(3,3-dimethylbutyrate) and 17-butyrate.

10. The method as defined by claim 7, wherein the corticosteroid is clobetasol-17-propionate.

11. The method as defined by claim 1, wherein the volatile solvent is selected from the group consisting of alkanols, alkylglycols, alkylketones and/or alkyl esters wherein the alkyl moieties thereof contain from 1 to 6 carbon atoms.

12. The method as defined by claim 11, wherein the volatile solvent is ethanol.

13. The method as defined by claim 1, wherein the film-forming silicone comprises at least one polyorganosiloxane elastomer.

14. The method as defined by claim 13, wherein the polyorganosiloxane elastomer is in a least one volatile silicone oil that is a linear or cyclic polyorganosiloxane oil having 2 to 10 silicon atoms.

15. The method as defined by claim 1, wherein said film-forming silicone is at a concentration of 20% to 90% by weight based on the total weight of the composition.

16. The method as defined by claim 1, wherein said volatile solvent is at a concentration of 1% to 50% by weight based on the total weight of the composition.

17. The method as defined by claim 1, wherein the composition is in form of a cream, a gel or an ointment.

18. The method as defined by claim 1, wherein the composition is substantially free of water.

19. The method as defined by claim 1, wherein the composition comprises:
isopropyl palmitate,
cyclopentasiloxane,
cyclomethicone 5/dimethicone crosspolymer,
calcitriol,
clobetasol-17-proprionate,
ethanol.

20. The method as defined by claim 19, wherein the composition comprises, in weight/weight of the composition:
isopropyl palmitate 0.5%-2%,
cyclopentasiloxane 10%-20%,
cyclomethicone 5/dimethicone crosspolymer 70%-80%,
calcitriol 0.0001%-0.0005%,
clobetasol-17-proprionate 0.01%-0.05%,
ethanol 5%-10%.

21. The method as defined by claim 1, comprising topically applying said composition onto the skin of an individual afflicted with a disorder of the skin.

22. The method as defined by claim 1, comprising topically applying said composition onto the affected skin area of an individual afflicted with psoriasis.

23. The method as defined by claim 1, comprising establishing a reservoir of said drug in the upper layers of the skin.

24. The method as defined by claim 23, said drug being supersaturated in the lower layers of the skin.

25. The method as defined by claim 24, comprising the controlled release of said drug at an essentially zero-order release rate.

26. A pharmaceutically active composition useful for the slow and sustained, controlled release of a drug into and through the skin, comprising at least one solubilized drug, at least one film-forming silicone, and at least one volatile solvent.

27. The pharmaceutically active composition as defined by claim 26, comprising solubilized vitamin D or a vitamin D analogue.

28. The pharmaceutically active composition as defined by claim 26, comprising a solubilized corticosteroid.

29. The pharmaceutically active composition as defined by claim 26, comprising at least one polyorganosiloxane elastomer.
